SEQUENCE LISTING (24 sequences)

SEQ ID NO: 1 - RNA, 21 nt, synthetic construct
  source 1..21: mol_type = other RNA
  modified_base 20..21: mod_base = OTHER; note = Thymine
tgtgtgtgtgtgtgtgtgttt

SEQ ID NO: 2 - RNA, 21 nt, synthetic construct
  source 1..21: mol_type = other RNA
  modified_base 20..21: mod_base = OTHER; note = deoxyadenosine
acacacacacacacacacaaa

SEQ ID NO: 3 - RNA, 21 nt, synthetic construct
  source 1..21: mol_type = other RNA
  modified_base 20..21: mod_base = OTHER; note = Thymine
gtgtgtgtgtgtgtgtgtgtt

SEQ ID NO: 4 - RNA, 21 nt, synthetic construct
  source 1..21: mol_type = other RNA
  modified_base 20..21: mod_base = OTHER; note = doxyadenosine
cacacacacacacacacacaa

SEQ ID NO: 5 - RNA, 21 nt, synthetic construct
  source 1..21: mol_type = other RNA
  modified_base 20..21: mod_base = OTHER; note = Thymine
tctctctctctctctctcttt

SEQ ID NO: 6 - RNA, 21 nt, synthetic construct
  source 1..21: mol_type = other RNA
  modified_base 20..21: mod_base = OTHER; note = deoxyadenosine
agagagagagagagagagaaa

SEQ ID NO: 7 - RNA, 21 nt, synthetic construct
  source 1..21: mol_type = other RNA
  modified_base 20..21: mod_base = OTHER; note = Thymine
ctctctctctctctctctctt

SEQ ID NO: 8 - RNA, 21 nt, synthetic construct
  source 1..21: mol_type = other RNA
  modified_base 20..21: mod_base = OTHER; note = deoxyadenosine
gagagagagagagagagagaa

SEQ ID NO: 9 - RNA, 21 nt, synthetic construct
  source 1..21: mol_type = other RNA
  modified_base 20..21: mod_base = OTHER; note = Thymine
tatatatatatatatatattt

SEQ ID NO: 10 - RNA, 21 nt, synthetic construct
  source 1..21: mol_type = other RNA
  modified_base 20..21: mod_base = OTHER; note = deoxyadenosine
atatatatatatatatataaa

SEQ ID NO: 11 - RNA, 21 nt, synthetic construct
  source 1..21: mol_type = other RNA
  modified_base 20..21: mod_base = OTHER; note = Thymine
atatatatatatatatatatt

SEQ ID NO: 12 - RNA, 21 nt, synthetic construct
  source 1..21: mol_type = other RNA
  modified_base 20..21: mod_base = OTHER; note = deoxyadenosine
tatatatatatatatatataa

SEQ ID NO: 13 - RNA, 21 nt, synthetic construct
  source 1..21: mol_type = other RNA
  modified_base 20..21: mod_base = OTHER; note = Thymine
gcgcgcgcgcgcgcgcgcgtt

SEQ ID NO: 14 - RNA, 21 nt, synthetic construct
  source 1..21: mol_type = other RNA
  modified_base 20..21: mod_base = OTHER; note = deoxyadenosine
cgcgcgcgcgcgcgcgcgcaa

SEQ ID NO: 15 - RNA, 21 nt, synthetic construct
  source 1..21: mol_type = other RNA
  modified_base 20..21: mod_base = OTHER; note = Thymine
cgcgcgcgcgcgcgcgcgctt

SEQ ID NO: 16 - RNA, 21 nt, synthetic construct
  source 1..21: mol_type = other RNA
  modified_base 20..21: mod_base = OTHER; note = deoxyadenosine
gcgcgcgcgcgcgcgcgcgaa

SEQ ID NO: 17 - RNA, 21 nt, synthetic construct
  source 1..21: mol_type = other RNA
  modified_base 20..21: mod_base = OTHER; note = Thymine
gagagagagagagagagagtt

SEQ ID NO: 18 - RNA, 21 nt, synthetic construct
  source 1..21: mol_type = other RNA
  modified_base 20..21: mod_base = OTHER; note = deoxyadenosine
ctctctctctctctctctcaa

SEQ ID NO: 19 - RNA, 21 nt, synthetic construct
  source 1..21: mol_type = other RNA
  modified_base 20..21: mod_base = OTHER; note = Thymine
agagagagagagagagagatt

SEQ ID NO: 20 - RNA, 21 nt, synthetic construct
  source 1..21: mol_type = other RNA
  modified_base 20..21: mod_base = OTHER; note = deoxyadenosine
tctctctctctctctctctaa

SEQ ID NO: 21 - RNA, 21 nt, synthetic construct
  source 1..21: mol_type = other RNA
  modified_base 20..21: mod_base = OTHER; note = Thymine
cacacacacacacacacactt

SEQ ID NO: 22 - RNA, 21 nt, synthetic construct
  source 1..21: mol_type = other RNA
  modified_base 20..21: mod_base = OTHER; note = deoxyadenosine
gtgtgtgtgtgtgtgtgtgaa

SEQ ID NO: 23 - RNA, 21 nt, synthetic construct
  source 1..21: mol_type = other RNA
  modified_base 20..21: mod_base = OTHER; note = Thymine
acacacacacacacacacatt

SEQ ID NO: 24 - RNA, 21 nt, synthetic construct
  source 1..21: mol_type = other RNA
  modified_base 20..21: mod_base = OTHER; note = deoxyadenosine
tgtgtgtgtgtgtgtgtgtaa

(19) United States
(12) Patent Application Publication
(10) Pub. No.: US 2025/0179497 A1
(43) Pub. Date: Jun. 5, 2025

(54) USE OF DINUCLEOTIDE REPEAT RNAS TO TREAT CANCER

(71) Applicant: Northwestern University, Evanston, IL (US)

(72) Inventors: Marcus Ernst Peter, Evanston, IL (US); Andrea Eveline Murmann, Evanston, IL (US)

(21) Appl. No.: 18/839,266

(22) PCT Filed: Feb. 16, 2023
(86) PCT No.: PCT/US2023/062759

§ 371 Date: Aug. 16, 2024

Related U.S. Application Data
(60) Provisional application No. 63/311,434, filed on Feb. 17, 2022.

(51) Int. Cl. C12N 15/113

(52) U.S. Cl. CPC C12N 15/1135; C12N 2310/14; C12N 2310/321

(57) ABSTRACT

Disclosed are polynucleotides, compositions, and methods related to RNA interference (RNAi). In particular, disclosed are toxic RNAi sequences comprising a dsRNA having a first strand, otherwise referred to as an “A” strand, and a second strand, otherwise referred to as a “B” strand and dinucleotide repeats. Also included are methods of using said complexes for killing cancer cells and treating cancer.

### CROSS-REFERENCE TO RELATED APPLICATIONS

The present application claims priority to U.S. Provisional Patent Application No. 63/311,434 that was filed Feb. 17, 2022, the entire contents of which are hereby incorporated by reference.

### STATEMENT REGARDING FEDERALLY SPONSORED RESEARCH OR DEVELOPMENT

This invention was made with government support under CA197450 awarded by the National Institutes of Health. The government has certain rights in the invention.

### SEQUENCE LISTING

A Sequence Listing accompanies this application and is submitted as an xml file of the sequence listing named “702581_02299.xml” which is 35,140 bytes in size and was created on Feb. 15, 2023. The sequence listing is electronically submitted via Patent Center and is incorporated herein by reference in its entirety.

### BACKGROUND

Apart from surgery, most current, clinically used cancer therapies are based on four approaches: chemotherapy, radiation therapy, targeted therapy, and immunotherapy. These therapies all suffer from several shortcomings. They are either toxic (chemotherapy, radiation therapy, and immunotherapy), prone to the acquisition of resistance (chemotherapy, radiation therapy, targeted therapy), and not curative for most cancers (chemotherapy, radiation therapy, targeted therapy, and immunotherapy). Successful treatments produce an objective response, but often extend life by only a few months. Therefore, there exists a need in the art for curative therapies with minimal side effects for the treatment of cancer.

### SUMMARY

In one aspect of the current disclosure, compositions are provided. In some embodiments, the compositions comprise: a dsRNA having a first strand, otherwise referred to as an “A” strand, and a second strand, otherwise referred to as a “B” strand,

5′-A01 A02 A03 A04 A05 A06 A07 A08 A09 A10 A11 A12 A13 A14 A15 A16 A17 A18 A19-3′
| | | | | | | | | | | | | | | | | | |
3′-B19 B18 B17 B16 B15 B14 B13 B12 B11 B10 B09 B08 B07 B06 B05 B04 B03 B02 B01-5′,

the dsRNA defined as follows: wherein A01 through A19 and B01 through B19 are any ribonucleotide selected from A, U, G, and C, provided that: (i) A01-A19 are complementary to B01-B19; (ii) the B strand comprises a dinucleotide repeat sequence (X₁X₂)ₙ, wherein X₁ and X₂ independently are selected from any ribonucleotide A, C, G, and U, and n is an integer from 3-9; or the A strand comprises a dinucleotide repeat sequence (X₁X₂)ₙ, wherein X₁ and X₂ independently are selected from any ribonucleotide A, C, G, and U, and n is an integer from 3-9. In some embodiments, the dinucleotide repeat is selected from: (CU)ₙ, (UC)ₙ, (AC)ₙ, (CA)ₙ, (AG)ₙ, (GA)ₙ, (GU)ₙ, and (UG)ₙ. In some embodiments, the dinucleotide repeat is selected from (UC)ₙ and (CU)ₙ. In some embodiments, the dsRNA further comprises nucleic acid overhangs on the 3′ end of either the A, B, or both A and B strands. In some embodiments, the nucleic acid overhangs are (dA)ₘ. In some embodiments, m=2. In some embodiments, the overhang is only present on the B strand. In some embodiments, the composition comprises modified nucleotides. In some embodiments, the A01 and A02 ribonucleotides or the B01 and B02 ribonucleotides comprise 2′-O-methylation modifications. In some embodiments, the dsRNA comprises a sequence selected from the group consisting of: SEQ ID NO: 1-24. In some embodiments, the dsRNA comprises SEQ ID NO: 17 or 18. In some embodiments, the composition comprises SEQ ID NO: 19 or 20.

In another aspect of the current disclosure, pharmaceutical compositions are provided. In some embodiments, the pharmaceutical compositions comprise a dsRNA having a first strand, otherwise referred to as an “A” strand, and a second strand, otherwise referred to as a “B” strand,

5′-A01 A02 A03 A04 A05 A06 A07 A08 A09 A10 A11 A12 A13 A14 A15 A16 A17 A18 A19-3′
| | | | | | | | | | | | | | | | | | |
3′-B19 B18 B17 B16 B15 B14 B13 B12 B11 B10 B09 B08 B07 B06 B05 B04 B03 B02 B01-5′,

the dsRNA defined as follows: wherein A01 through A19 and B01 through B19 are any ribonucleotide selected from A, U, G, and C, provided that: (i) A01-A19 are complementary to B01-B19; (ii) the B strand comprises a dinucleotide repeat sequence (X₁X₂)ₙ, wherein X₁ and X₂ independently are selected from any ribonucleotide A, C, G, and U, and n is an integer from 3-9; or the A strand comprises a dinucleotide repeat sequence (X₁X₂)ₙ, wherein X₁ and X₂ independently are selected from any ribonucleotide A, C, G, and U, and n is an integer from 3-9 and a pharmaceutically acceptable carrier. In some embodiments, the dinucleotide repeat is selected from: (CU)ₙ, (UC)ₙ, (AC)ₙ, (CA)ₙ, (AG)ₙ, (GA)ₙ, (GU)ₙ, and (UG)ₙ. In some embodiments, the dinucleotide repeat is selected from (UC)ₙ and (CU)ₙ. In some embodiments, the dsRNA further comprises nucleic acid overhangs on the 3′ end of either the A, B, or both A and B strands. In some embodiments, the nucleic acid overhangs are (dA)ₘ. In some embodiments, m=2. In some embodiments, the overhang is only present on the B strand. In some embodiments, the composition comprises modified nucleotides. In some embodiments, the A01 and A02 ribonucleotides or the B01 and B02 ribonucleotides comprise 2′-O-methylation modifications. In some embodiments, the dsRNA comprises a sequence selected from the group consisting of: SEQ ID NO: 1-24. In some embodiments, the dsRNA comprises SEQ ID NO: 17 or 18. In some embodiments, the composition comprises SEQ ID NO: 19 or 20.

In another aspect of the current disclosure, methods of treating a cell proliferative disease or disorder in a subject in need thereof are provided. In some embodiments, the methods comprise administering an effective amount of a pharmaceutical composition comprising a dsRNA having a first strand, otherwise referred to as an “A” strand, and a second strand, otherwise referred to as a “B” strand,

5′-A01 A02 A03 A04 A05 A06 A07 A08 A09 A10 A11 A12 A13 A14 A15 A16 A17 A18 A19-3′
| | | | | | | | | | | | | | | | | | |
3′-B19 B18 B17 B16 B15 B14 B13 B12 B11 B10 B09 B08 B07 B06 B05 B04 B03 B02 B01-5′,

the dsRNA defined as follows: wherein A01 through A19 and B01 through B19 are any ribonucleotide selected from A, U, G, and C, provided that: (i) A01-A19 are complementary to B01-B19; (ii) the B strand comprises a dinucleotide repeat sequence (X₁X₂)ₙ, wherein X₁ and X₂ independently are selected from any ribonucleotide A, C, G, and U, and n is an integer from 3-9; or the A strand comprises a dinucleotide repeat sequence (X₁X₂)ₙ, wherein X₁ and X₂ independently are selected from any ribonucleotide A, C, G, and U, and n is an integer from 3-9 and a pharmaceutically acceptable carrier to the subject to treat the cell proliferative disease or disorder. In some embodiments, the dinucleotide repeat is selected from: (CU)ₙ, (UC)ₙ, (AC)ₙ, (CA)ₙ, (AG)ₙ, (GA)ₙ, (GU)ₙ, and (UG)ₙ. In some embodiments, the dinucleotide repeat is selected from (UC)ₙ and (CU)ₙ. In some embodiments, the dsRNA further comprises nucleic acid overhangs on the 3′ end of either the A, B, or both A and B strands. In some embodiments, the nucleic acid overhangs are (dA)ₘ. In some embodiments, m=2. In some embodiments, the overhang is only present on the B strand. In some embodiments, the composition comprises modified nucleotides. In some embodiments, the A01 and A02 ribonucleotides or the B01 and B02 ribonucleotides comprise 2′-O-methylation modifications. In some embodiments, the dsRNA comprises a sequence selected from the group consisting of: SEQ ID NO: 1-24. In some embodiments, the dsRNA comprises SEQ ID NO: 17 or 18. In some embodiments, the composition comprises SEQ ID NO: 19 or 20. In some embodiments, the cell proliferative disease or disorder is cancer. In some embodiments, the cancer is selected from lung cancer, prostate cancer, ovarian cancer, colon cancer, and hepatocellular carcinoma.

In another aspect of the current disclosure, methods of killing a cancer cell are provided. In some embodiments, the methods comprise contacting a composition comprising a dsRNA having a first strand, otherwise referred to as an “A” strand, and a second strand, otherwise referred to as a “B” strand,

5′-A01 A02 A03 A04 A05 A06 A07 A08 A09 A10 A11 A12 A13 A14 A15 A16 A17 A18 A19-3′
| | | | | | | | | | | | | | | | | | |
3′-B19 B18 B17 B16 B15 B14 B13 B12 B11 B10 B09 B08 B07 B06 B05 B04 B03 B02 B01-5′,

the dsRNA defined as follows: wherein A01 through A19 and B01 through B19 are any ribonucleotide selected from A, U, G, and C, provided that: (i) A01-A19 are complementary to B01-B19; (ii) the B strand comprises a dinucleotide repeat sequence (X₁X₂)ₙ, wherein X₁ and X₂ independently are selected from any ribonucleotide A, C, G, and U, and n is an integer from 3-9; or the A strand comprises a dinucleotide repeat sequence (X₁X₂)ₙ, wherein X₁ and X₂ independently are selected from any ribonucleotide A, C, G, and U, and n is an integer from 3-9 to the cancer cell. In some embodiments, the dinucleotide repeat is selected from: (CU)ₙ, (UC)ₙ, (AC)ₙ, (CA)ₙ, (AG)ₙ, (GA)ₙ, (GU)ₙ, and (UG)ₙ. In some embodiments, the dinucleotide repeat is selected from (UC)ₙ and (CU)ₙ. In some embodiments, the dsRNA further comprises nucleic acid overhangs on the 3′ end of either the A, B, or both A and B strands. In some embodiments, the nucleic acid overhangs are (dA)ₘ. In some embodiments, m=2. In some embodiments, the overhang is only present on the B strand. In some embodiments, the composition comprises modified nucleotides. In some embodiments, the A01 and A02 ribonucleotides or the B01 and B02 ribonucleotides comprise 2′-O-methylation modifications. In some embodiments, the dsRNA comprises a sequence selected from the group consisting of: SEQ ID NO: 1-24. In some embodiments, the dsRNA comprises SEQ ID NO: 17 or 18. In some embodiments, the composition comprises SEQ ID NO: 19 or 20. In some embodiments, the cancer cell is, or is derived from lung cancer, prostate cancer, ovarian cancer, colon cancer, or hepatocellular carcinoma.

### BRIEF DESCRIPTION OF THE FIGURES

FIGS. 1A and 1B: siRNA screen identifies most toxic dinucleotide repeat (DNR) containing siRNAs. (A) Structure and sequences of all possible DNRs that are comprised of more than one nucleotide. The position of two 2′-O-methylation modifications is indicated by a red X at position 1 and 2 of the passenger strand. (B) Viability of the human ovarian cancer cells line HeyA8 and the mouse hepatocellular carcinoma cell line M565 96 hrs after transfection with 10 nM of siRNAs comprised of the indicated DNRs. Screen and viability assay was performed exactly as described in (2). Loss of viability of 50% or more is indicated by orange columns and of 75% or more by red columns.

FIGS. 2A and 2B: Effect of the different DNR based siRNAs on cell growth of HeyA8 cells. Change in confluency of HeyA8 cells reverse transfected with either 10 nM (A) or 1 nM (B) of the indicated siRNAs.

FIGS. 3A, 3B, 3C, and 3D: Effect of the DNR based siRNAs on cell growth of different cancers. Change in confluency of H460 (A), H23 (B), H522 (C), and PC3 (D) cells reverse transfected with either 1 nM (A, D) or 10 nM siRNAs (A, B, C). siL3 is a FasL derived siRNA that kills cells through 6mer seed toxicity (1).

FIG. 4: DNR based siRNA kill cancer cells through RNAi. Change in confluency of HCT116 wt (A) and HCT116 Ago1/2/3 triple knockout cells (B) reverse transfected with 1 nM siRNAs.

FIG. 5: Frequency of different DNRs in the coding regions or 3′ UTRs of human and mouse genes. Only DNRs were scored that are 19 nts or longer. For each gene the longest transcript was analyzed.

FIGS. 6A, 6B, 6C, and 6D: Identification of most significantly downregulated GA DNR containing target genes in HeyA8 cells transfected with siUC. HeyA8 cells were transfected with either siNT1 or siUC (in duplicate) at 1 nM and after 48 hrs subjected to RNAseq analysis. Only genes with at least 1000 normalized reads in the siNT1 transfected controls downregulated at least 1.5-fold with an adjusted p-value of <0.05 were included in the analysis. These were 62 genes. (A) The top 25 most highly downregulated genes of this analysis containing GA DNRs of 10 nts or longer ranked according to the highest fold downregulation. Genes highlighted in green are essential survival genes either in our original list of SG (1) or in the list of DepMap essential survival genes obtained by screening 1840 human cell lines (DepMap.org). The repeat length in nts detected is shown for the top 10 genes as well as the location of the DNR in the mRNA (either open reading frame (ORF) or 3′ untranslated region (3′UTR)). (B) Read number of individual samples of the top 5 genes in A. T-test p-values between the two duplicates are shown. (C) Venn diagram showing the overlap between the combined SGs and the 62 significantly downregulated genes. (D) GSEA of all deregulated original (left) and DepMap (right) SGs ranked from highest (left) to lowest (right) downregulation (siUC versus siNT1).

FIGS. 7A, 7B, and 7C: Identification of most significantly downregulated AG DNR containing target genes in HeyA8 cells transfected with siCU. HeyA8 cells were transfected with either siNT1 or siUC (in duplicate) at 1 nM and after 48 hrs subjected to RNAseq analysis. Only genes with at least 1000 normalized reads in the siNT1 transfected controls downregulated at least 1.5-fold with an adjusted p-value of <0.05 were included in the analysis. These were 43 genes. (A) The top 25 most highly downregulated genes of this analysis containing AG DNRs of 10 nts or longer ranked according to the highest fold downregulation. Genes highlighted in green are essential survival genes either in our original list of SG (1) or in the list of DepMap essential survival genes obtained by screening 1840 human cell lines (DepMap.org). The repeat length in nts detected is shown for the top 10 genes as well as the location of the DNR in the mRNA (either open reading frame (ORF) or 3′ untranslated region (3′UTR)). (B) Read number of individual samples of the top 5 genes in A. T-test p-values between the two duplicates are shown. (C) Venn diagram showing the overlap between the combined SGs and the 43 significantly downregulated genes.

FIGS. 8A and 8B: UC and CU DNR containing siRNAs target widely overlapping set genes consistent with targeting GA containing DNRs. (A) Regression analysis of all genes deregulated in HeyA8 cells 48 hours after transfection with either siUC (X axis) or siCU (Y axis). (B) Venn diagram of the significantly expressed (at least 1000 reads in the control) and deregulated (>1.5 fold and padj<0.05) AG DNR (for the siCU analysis) or GA DNR (for the siUC analysis) containing genes.

FIG. 9: Enrichment of GA/AG DNR target mRNAs in biotin-streptavidin pulled down RNA normalized to input RNA in HeyA8 cells transfected with 10 nM Bi-siNT1 Bi-siUC, or Bi-siCU for 24 hrs by RNASeq. Shown are the top 5 most downregulated siUC (top left)/siCU (top right) targeted genes (see FIG. 6A/7A) and GAPDH as negative control which does not contain a GA/GA DNR. 5 genes that do not contain either GA or AG DRNs targeted motifs but are expressed at similar levels as the target genes (not shown) are shown as controls (bottom panels). Each column represents ±SD of three replicates. Students t-test was used to calculate p-value. * p<0.05, *** p<0.0000001. ns, not significant.

### DETAILED DESCRIPTION

The present invention is described herein using several definitions, as set forth below and throughout the application.

Unless defined otherwise, all technical and scientific terms used herein have the same meaning as commonly understood by one of skill in the art to which the invention pertains. All definitions, as defined and used herein, should be understood to control over dictionary definitions, definitions in documents incorporated by reference, and/or ordinary meanings of the defined terms.

Unless otherwise specified or indicated by context, the terms “a”, “an”, and “the” should be interpreted to mean “one or more.” For example, “an shRNA” or “an siRNA” should be interpreted to mean “one or more shRNA's” and “one or more siRNA's,” respectively

As used herein, “about,” “approximately,” “substantially,” and “significantly” will be understood by persons of ordinary skill in the art and will vary to some extent on the context in which they are used. If there are uses of these terms which are not clear to persons of ordinary skill in the art given the context in which they are used, “about” and “approximately” should be interpreted to mean plus or minus ≤10% of the particular term and “substantially” and “significantly” should be interpreted to mean plus or minus >10% of the particular term.

As used herein, the terms “include” and “including” should be interpreted to have the same meaning as the terms “comprise” and “comprising” in that these latter terms are “open” transitional terms that do not limit claims only to the recited elements succeeding these transitional terms. The term “consisting of,” while encompassed by the term “comprising,” should be interpreted as a “closed” transitional term that limits claims only to the recited elements succeeding this transitional term. The term “consisting essentially of,” while encompassed by the term “comprising,” should be interpreted as a “partially closed” transitional term which permits additional elements succeeding this transitional term, but only if those additional elements do not materially affect the basic and novel characteristics of the claim.

A range includes each individual member. Thus, for example, a group having 1-3 members refers to groups having 1, 2, or 3 members.

It should also be understood that, unless clearly indicated to the contrary, in any methods claimed herein that include more than one step or act, the order of the steps or acts of the method is not necessarily limited to the order in which the steps or acts of the method are recited.

The modal verb “may” refers to the preferred use or selection of one or more options or choices among the several described embodiments or features contained within the same. Where no options or choices are disclosed regarding a particular embodiment or feature contained in the same, the modal verb “may” refers to an affirmative act regarding how to make or use an aspect of a described embodiment or feature contained in the same, or a definitive decision to use a specific skill regarding a described embodiment or feature contained in the same. In this latter context, the modal verb “may” has the same meaning and connotation as the auxiliary verb “can.”

As used herein, a “subject” may be interchangeable with “patient” or “individual” and means an animal, which may be a human or non-human animal, in need of treatment.

A “subject in need of treatment” may include a subject having a disease, disorder, or condition that can be treated by administering to the subject one or more therapeutic RNAs as disclosed herein. A subject in need thereof may include a subject having or at risk for developing a cell proliferative disease or disorder such as cancer. A subject in need thereof may include, but is not limited to, a subject having or at risk for developing any of adenocarcinoma, leukemia, lymphoma, melanoma, myeloma, sarcoma, and teratocarcinoma, (including cancers of the adrenal gland, bladder, bone, bone marrow, brain, breast, cervix, gall bladder, ganglia, gastrointestinal tract, heart, kidney, liver, lung, muscle, ovary, pancreas, parathyroid, prostate, skin, testis, thymus, and uterus). In some embodiments, a subject in need of treatment may include a subject suffering from lung cancer, prostate cancer, ovarian cancer, or hepatocellular carcinoma. As such, methods of treating cancers are contemplated herein, including methods of treating cancers selected from, but not limited to any of adenocarcinoma, leukemia, lymphoma, melanoma, myeloma, sarcoma, and teratocarcinoma, lung cancer, prostate cancer, ovarian cancer, and hepatocellular carcinoma, (including cancers of the adrenal gland, bladder, bone, bone marrow, brain, breast, cervix, gall bladder, ganglia, gastrointestinal tract, heart, kidney, liver, lung, muscle, ovary, pancreas, parathyroid, prostate, skin, testis, thymus, and uterus).

As used herein, a “toxic RNA” refers to an RNA molecule that induces cell death via RNA interference (RNAi) when the RNA molecule is expressed in a cell. Toxic RNAs may include, but are not limited to toxic shRNA, toxic siRNA (which may have been processed via Dicer from a corresponding shRNA), toxic pre-miRNA which may artificial or engineered pre-miRNA, and/or toxic miRNA (which may have been processed via Dicer from a corresponding pre-miRNA). Toxic RNAs have been disclosed in the art. (See U.S. Published application Nos. 20180251762 and 20180320187, the contents of which are incorporated herein by reference in their entireties).

As used herein, the terms “silencing” and “inhibiting the expression of” refer to at least partial suppression of the expression of a target gene, for example, as manifested by a reduction of mRNA associated with the target gene.

As used herein, the phrase “effective amount” shall mean that drug dosage that provides the specific pharmacological response for which the drug is administered in a significant number of patients in need of such treatment. An effective amount of a drug that is administered to a particular patient in a particular instance will not always be effective in treating the conditions/diseases described herein, even though such dosage is deemed to be a therapeutically effective amount by those of skill in the art.

### Polynucleotides

The disclosed technology relates to nucleic acid and the use of nucleic acid for treating diseases and disorders. The terms “nucleic acid” and “oligonucleotide,” as used herein, refer to polydeoxyribonucleotides (containing 2-deoxy-ribose), polyribonucleotides (containing ribose), and to any other type of polynucleotide that is an N glycoside of a purine or pyrimidine base. As used herein, the terms “A,” “T,” “C”, “G” and “U” refer to adenine, thymine, cytosine, guanine, uracil as a nucleotide base, respectively. There is no intended distinction in length between the terms “nucleic acid,” “oligonucleotide,” and “polynucleotide,” and these terms will be used interchangeably. These terms refer only to the primary structure of the molecule. Thus, these terms include double- and single-stranded DNA, as well as double- and single-stranded RNA. For use in the present invention, an oligonucleotide also can comprise nucleotide analogs in which the base, sugar or phosphate backbone is modified as well as non-purine or non-pyrimidine nucleotide analogs.

The disclosed polynucleotides may include a fragment of a reference polynucleotide. As used herein, a “fragment” of a polynucleotide is a portion of a polynucleotide sequence which is identical in sequence to but shorter in length than a reference sequence. A fragment may comprise up to the entire length of the reference sequence, minus at least one nucleotide. For example, a fragment may comprise from 5 to 1000 contiguous nucleotides of a reference polynucleotide. In some embodiments, a fragment may comprise at least 5, 10, 15, 20, 25, 30, 40, 50, 60, 70, 80, 90, 100, 150, 250, or 500 contiguous nucleotides of a reference polynucleotide; in other embodiments a fragment may comprise no more than 5, 10, 15, 20, 25, 30, 40, 50, 60, 70, 80, 90, 100, 150, 250, or 500 contiguous nucleotides of a reference polynucleotide; in further embodiments a fragment may comprise a range of contiguous nucleotides of a reference polynucleotide bounded by any of the foregoing values (e.g. a fragment comprising 20-50 contiguous nucleotides of a reference polynucleotide). Fragments may be preferentially selected from certain regions of a molecule. The term “at least a fragment” encompasses the full-length polynucleotide. A “variant,” “mutant,” or “derivative” of a reference polynucleotide sequence may include a fragment of the reference polynucleotide sequence.

The disclosed polynucleotides may include a deletion relative to a reference polynucleotide. As used herein, a “deletion” refers to a change in a reference nucleotide sequence that results in the absence of one or more nucleotide residues. For example, a deletion may remove at least 1, 2, 3, 4, 5, 10, 20, 50, 100, or 200 nucleotide residues or a range of nucleotide residues bounded by any of these values (e.g., a deletion of 5-10 nucleotides). A deletion may include an internal deletion or a terminal deletion (e.g., a 5′-terminal truncation or a 3′-terminal truncation of a reference polynucleotide). A “variant” of a reference nucleotide sequence may include a deletion relative to the reference polynucleotide sequence.

The disclosed polynucleotides may include an insertion or an addition relative to a reference polynucleotide. As used herein, “insertion” and “addition” refer to changes in a nucleotide sequence resulting in the addition of one or more nucleotide residues. An insertion or addition may refer to 1, 2, 3, 4, 5, 10, 20, 30, 40, 50, 60, 70, 80, 90, 100, 150, or 200 nucleotide residues or a range of nucleotide residues bounded by any of these values (e.g., an insertion or addition of 5-10 nucleotides). A “variant” of a reference polynucleotide sequence may include an insertion or addition relative to the reference polynucleotide sequence.

Regarding polynucleotide sequences, percent identity may be measured over the length of an entire defined polynucleotide sequence, for example, as defined by a particular SEQ ID number, or may be measured over a shorter length, for example, over the length of a fragment taken from a larger, defined sequence, for instance, a fragment of at least 20, at least 30, at least 40, at least 50, at least 70, at least 100, or at least 200 contiguous nucleotides. Such lengths are exemplary only, and it is understood that any fragment length supported by the sequences shown herein, in the tables, figures, or Sequence Listing, may be used to describe a length over which percentage identity may be measured.

Regarding polynucleotide sequences, “variant,” “mutant,” or “derivative” may be defined as a nucleic acid sequence having at least 50% sequence identity to the particular nucleic acid sequence over a certain length of one of the nucleic acid sequences using blastn with the “BLAST 2 Sequences” tool available at the National Center for Biotechnology Information's website. (See Tatiana A. Tatusova, Thomas L. Madden (1999), “Blast 2 sequences—a new tool for comparing protein and nucleotide sequences”, FEMS Microbiol Lett. 174:247-250). Such a pair of nucleic acids may show, for example, at least 20%, at least 30%, at least 40%, at least 50%, at least 60%, at least 70%, at least 80%, at least 85%, at least 90%, at least 91%, at least 92%, at least 93%, at least 94%, at least 95%, at least 96%, at least 97%, at least 98%, or at least 99% or greater sequence identity over a certain defined length.

A “recombinant nucleic acid” is a sequence that is not naturally occurring or has a sequence that is made by an artificial combination of two or more otherwise separated segments of sequence. This artificial combination is often accomplished by chemical synthesis or, more commonly, by the artificial manipulation of isolated segments of nucleic acids, e.g., by genetic engineering techniques known in the art. The term recombinant includes nucleic acids that have been altered solely by addition, substitution, or deletion of a portion of the nucleic acid. Frequently, a recombinant nucleic acid may include a nucleic acid sequence operably linked to a promoter sequence. Such a recombinant nucleic acid may be part of a vector that is used, for example, to transform a cell.

The nucleic acids disclosed herein may be “substantially isolated or purified.” The term “substantially isolated or purified” refers to a nucleic acid that is removed from its natural environment, and is at least 60% free, preferably at least 75% free, and more preferably at least 90% free, even more preferably at least 95% free from other components with which it is naturally associated.

Oligonucleotides can be prepared by any suitable method, including direct chemical synthesis by a method such as the phosphotriester method of Narang et al., 1979, Meth. Enzymol. 68:90-99; the phosphodiester method of Brown et al., 1979, Meth. Enzymol. 68:109-151; the diethylphosphoramidite method of Beaucage et al., 1981, Tetrahedron Letters 22:1859-1862; and the solid support method of U.S. Pat. No. 4,458,066, each incorporated herein by reference. A review of synthesis methods of conjugates of oligonucleotides and modified nucleotides is provided in Goodchild, 1990, Bioconjugate Chemistry 1 (3): 165-187, incorporated herein by reference.

The term “promoter” as used herein refers to a cis-acting DNA sequence that directs RNA polymerase and other trans-acting transcription factors to initiate RNA transcription from the DNA template that includes the cis-acting DNA sequence. Promoters may include inducible promoter, which are promoters that can be induced to function in the presence of an effector molecule as known in the art.

As used herein, the term “complementary” in reference to a first polynucleotide sequence and a second polynucleotide sequence means that the first polynucleotide sequence will base-pair exactly with the second polynucleotide sequence throughout a stretch of nucleotides without mismatch. The term “cognate” may in reference to a first polynucleotide sequence and a second polynucleotide sequence means that the first polynucleotide sequence will base-pair with the second polynucleotide sequence throughout a stretch of nucleotides but may include one or more mismatches within the stretch of nucleotides. As used herein, the term “complementary” may refer to the ability of a first polynucleotide to hybridize with a second polynucleotide due to base-pair interactions between the nucleotide pairs of the first polynucleotide and the second polynucleotide (e.g., A:T, A:U, C:G, G:C, G:U, T:A, U:A, and U:G).

As used herein, the term “complementarity” may refer to a sequence region on an anti-sense strand that is substantially complementary to a target sequence but not fully complementary to a target sequence. Where the anti-sense strand is not fully complementary to the target sequence, mismatches may be optionally present in the terminal regions of the anti-sense strand or elsewhere in the anti-sense strand. If mismatches are present, optionally the mismatches may be present in terminal region or regions of the anti-sense strand (e.g., within 6, 5, 4, 3, or 2 nucleotides of the 5′ and/or 3′ terminus of the anti-sense strand).

The term “hybridization,” as used herein, refers to the formation of a duplex structure by two single-stranded nucleic acids due to complementary base pairing. Hybridization can occur between fully complementary nucleic acid strands or between “substantially complementary” nucleic acid strands that contain minor regions of mismatch. Conditions under which hybridization of fully complementary nucleic acid strands is strongly preferred are referred to as “stringent hybridization conditions” or “sequence-specific hybridization conditions.” Stable duplexes of substantially complementary sequences can be achieved under less stringent hybridization conditions; the degree of mismatch tolerated can be controlled by suitable adjustment of the hybridization conditions. Those skilled in the art of nucleic acid technology can determine duplex stability empirically considering a number of variables including, for example, the length and base pair composition of the oligonucleotides, ionic strength, and incidence of mismatched base pairs, following the guidance provided by the art (see, e.g., Sambrook et al., 1989, Molecular Cloning-A Laboratory Manual, Cold Spring Harbor Laboratory, Cold Spring Harbor, New York; Wetmur, 1991, Critical Review in Biochem. and Mol. Biol. 26(3/4):227-259; and Owczarzy et al., 2008, Biochemistry, 47:5336-5353, which are incorporated herein by reference).

As used herein, the term “double-stranded RNA” (“dsRNA”) refers to a complex of ribonucleic acid molecules having a duplex structure comprising two anti-parallel and substantially complementary nucleic acid strands.

As used herein, the term “nucleotide overhang” refers to an unpaired nucleotide or nucleotides that extend from the 5′-end or 3′-end of a duplex structure of a dsRNA when a 5′-end of one strand of the dsRNA extends beyond the 3′-end of the other strand, or when a 3′-end of one strand of the dsRNA extends beyond the 5′-end of the other strand. A nucleotide overhang may include ribonucleotides and/or deoxyribonucleotide (e.g., dAdA or TT).

As used herein, the term “blunt” refers to a dsRNA in which there are no unpaired nucleotides at the 5′-end and/or the 3′-end of the dsRNA (i.e., no nucleotide overhang at the 5′-end or the 3′-end). A “blunt ended” dsRNA is a dsRNA that has no nucleotide overhang at the 5′-end or the 3′-end of the dsRNA molecule.

As used herein, the term “anti-sense strand” refers to a strand of a dsRNA which includes a region that is substantially complementary to a target sequence (i.e., where the target sequence has a sequence corresponding to the sense strand).

As used herein, the term “sense strand,” refers to the strand of a dsRNA that includes a region that is substantially complementary to a region of the anti-sense strand and that includes a region that substantially corresponds to a region of the target sequence.

As used herein, RNAi active sequences may include “siRNA” and “shRNA” and dsRNA that is processed by nucleases to provide siRNA and/or shRNA. The term “siRNA” refers to a “small interfering RNA” and the term “shRNA” refers to “short hairpin RNA.” RNA interference (RNAi) refers to the process of sequence-specific post-transcriptional gene silencing in a cell or an animal mediated by siRNA and/or shRNA.

As used herein, the term “siRNA targeted against mRNA” refers to siRNA specifically promote degradation of the targeted mRNA via sequence-specific complementary multiple base pairings (e.g., at least 6 contiguous base-pairs between the siRNA and the target mRNA at optionally at least 7, 8, 9, 10, 11, 12, 13, 14, 15, 16, 17, 18, 19, 20, 21, 22, or 23 contiguous base-pairs between the siRNA and the target mRNA).

As used herein, RNAi active sequences may include “pre-miRNA” and “miRNA” and dsRNA that is processed to provide pre-miRNA and miRNA. The term “pre-miRNA” refers to a “pre-micro RNA” and the term “miRNA” refers to “micro RNA.” RNA interference (RNAi) refers to the process of sequence-specific post-transcriptional gene silencing in a cell or an animal mediated by pre-miRNA and/or miRNA.

The terms “target, “target sequence”, “target region”, and “target nucleic acid,” as used herein, are synonymous and refer to a region or sequence of a nucleic acid which may be selected as a sequence to which the anti-sense strand of siRNA or shRNA is substantially complementary to and hybridizes to as discussed herein. A target sequence may refer to a contiguous portion of a nucleotide sequence of an mRNA molecule of a particular gene, including but not limited to, genes that are essential for survival and/or growth of cells and in particular cancer cells. The target sequence of a siRNA refers to a mRNA sequence of a gene that is targeted by the siRNA due to complementarity between the anti-sense strand of the siRNA and the mRNA sequence and to which the anti-sense strand of the siRNA hybridizes when brought into contact with the mRNA sequence.

As used herein, the term “transfecting” means “introducing into a cell” a molecule, which may include a polynucleotide molecule such as dsRNA. When referring to a dsRNA, transfecting means facilitating uptake or absorption into the cell, as is understood by the skilled person. Absorption or uptake of dsRNA can occur or may be facilitated through passive diffusive or active cellular processes, or through the use of auxiliary agents or devices. Transfection into a cell includes methods known in the art such as electroporation and lipofection. However, the meaning of the term “transfection” is not limited to introducing molecules into cells in vitro. As contemplated herein, a dsRNA also may be “introduced into a cell,” where the cell is part of a living organism. For example, for in vivo delivery, a dsRNA may be injected into a tissue site or may be administered systemically.

### RNA Interference

The mechanism of action of RNA interference (RNAi) is understood by the skilled person. Interfering RNA (RNAi) generally refers to process that utilizes a single-stranded RNA (ssRNA) or double-stranded RNA (dsRNA) to inhibit expression of a target. The dsRNA is capable of targeting specific messenger RNA (mRNA) and silencing (i.e., inhibiting) the expression of a target gene. During this process, dsRNA (which may include shRNA or pre-miRNA) is enzymatically processed into short-interfering RNA (siRNA) duplexes or miRNA duplexes by a nuclease called Dicer. The anti-sense strand of the siRNA duplex or miRNA duplex (referred to as the “guide strand”) is then incorporated into a cytoplasmic complex of proteins (RNA-induced silencing complex or RISC). The sense strand of the siRNA duplex of miRNA duplex (referred to as the “passenger strand”) is degraded. The RISC complex containing the anti-sense siRNA strand or anti-sense miRNA strand binds mRNA which has a sequence complementary to the anti-sense strand-allowing complementary base-pairing between the anti-sense strand and the sense mRNA molecule. The mRNA molecule is then specifically cleaved by an enzyme (RNase) associated with RISC called Argonaut 2 (Ago2) resulting in specific gene silencing. For gene silencing or knock down (i.e., mRNA cleavage) to occur, anti-sense RNA has to become incorporated into the RISC. This represents an efficient process that occurs in nucleated cells during regulation of gene expression.

In particular, siRNA-mediated RNA interference may be considered to involve two-steps: (i) an initiation step, and (ii) an effector step. In the first step, input siRNA is processed into small fragments by Dicer. These small fragments are ˜21-23-nucleotide in length and are called “guide RNAs.” The guide RNAs can be incorporated into the protein-RNA RISC complex which is capable of degrading mRNA. As such, the RISC complex acts in the second effector step to destroy mRNAs that are recognized by the guide RNAs through base-pairing interactions via Ago2. RNA interference may be considered to involve the introduction by any means of double stranded RNA into a cell which triggers events that cause the degradation of a target RNA, and as such may be considered to be a form of post-transcriptional gene silencing. The skilled person understands how to prepare and utilize RNA molecules in RNAi. (See, e.g., Hammond et al., Nature Rev Gen 2:110-119 (2001); and Sharp, Genes Dev 15:485-490 (2001), the contents of which are incorporate herein by reference in their entireties).

### Death Induced by Survival Gene Elimination

Previously, the inventors disclosed toxic RNAs that silence expression of one or more mRNAs of essential genes that are required for survival and growth of cells such as cancer cells. The disclosed toxic RNA molecules silence the expression of multiple mRNAs of essential genes that are required for survival and growth of cells such as cancer cells through a process similar to the process called “death-induced by survival gene elimination” or “DISE.”

For purposes of this application, the anti-sense strand of the siRNA may comprise a contiguous nucleotide sequence, where the base sequence of the anti-sense strand has substantial or complete sequence complementarity to the base sequence of a contiguous nucleotide sequence of corresponding length contained in an mRNA sequence of the targeted mRNA (e.g., in a non-coding 3′-end of an mRNA sequence). Substantial complementary permits some nucleotide mismatches (i.e., non-pairing nucleotides) and as such, the anti-sense strand of the siRNA need not have full complementarity.

In some embodiments, at least a portion of an anti-sense strand of an siRNA molecule comprises or consists of a sequence that is 100% complementary to a target sequence or a portion thereof. In another embodiment, at least a portion of an anti-sense strand of an siRNA molecule comprises or consists of a sequence that is at least about 90%, 95%, or 99% complementary to a target sequence or a portion thereof. For purposes of this application, the anti-sense strand of the siRNA molecule preferably comprises or consists of a sequence that specifically hybridizes to a target sequence or a portion thereof so as to inhibit expression of the target mRNA.

Methods for preparing and isolating siRNA also are known in the art. (See, e.g., Sambrook et al., Molecular Cloning, A Laboratory Manual (2.sup.nd Ed., 1989), the content of which is incorporated herein by reference in its entirety). The disclosed siRNA may be chemically synthesized, using any of a variety of techniques known in the art. The disclosed siRNA may include modifications, for example, modifications that stabilize the siRNA and/or protect the siRNA from degradation via endonucleases and/or exonucleases. In some embodiments, the disclosed siRNA may include nucleic acid protecting and coupling groups, such as dimethoxytrityl at the 5′-end and/or phosphoramidites at the 3′-end.

In one embodiment, the disclosed dsRNAs comprise a double stranded region of about 15 to about 30 nucleotides in length. Preferably, the disclosed RNAs are about 20-25 nucleotides in length. The disclosed RNAs of the present invention are capable of silencing the expression of a target sequence in vitro and in vivo.

In one embodiment, the dsRNA disclosed herein comprises a hairpin loop structure and may be referred to as shRNA which may be processed to a siRNA. In another embodiment, the dsRNA or siRNA has an overhang on its 3′ or 5′ ends relative to the target RNA which is to be cleaved. The overhang may be 2-10 nucleotides long. In one embodiment, the dsRNA or siRNA does not have an overhang (i.e., the dsRNA or siRNA has blunt ends).

In another embodiment, the disclosed dsRNA molecules (e.g., siRNA molecules) may contain one or more modified nucleotides, including one or more modified nucleotides at the 5′ and/or 3′ terminus of the RNA molecules. In yet another embodiment, the disclosed RNA molecules may comprise one, two, three four or more modified nucleotides in the double-stranded region. Exemplary modified nucleotides may include but are not limited to, modified nucleotides such as 2′-O-methyl (2′OMe) nucleotides, 2′-deoxy-2′-fluoro (2′F) nucleotides, 2′-deoxy nucleotides, 2′-O-(2-methoxyethyl) (MOE) nucleotides, and the like. The preparation of modified siRNA is known by one skilled in the art. In some embodiments, the disclosed dsRNA molecules include one or more modified nucleotides at the 5′-terminus of the passenger strand of the dsRNA that prevent incorporation of the passenger strand into RISC. (See, e.g., Walton et al., Minireview: “Designing highly active siRNAs for therapeutic applications,” the FEBS Journal, 277 (2010) 4806-4813).

In some embodiments, the disclosed siRNA molecules are capable of silencing one or more target mRNAs and may reduce expression of the one or more target mRNAs by at least about 20%, 30%, 40%, 50%, 60%, 70%, 80%, 90%, or 100% relative to a control siRNA molecule (e.g., a molecule not exhibiting substantial complementarity with the target mRNA). As such, in some embodiments, the presently disclosed siRNA molecules targeting the mRNA of essential genes may be used to down-regulate or inhibit the expression of essential genes (e.g., by at least about 20%, 30%, 40%, 50%, 60%, 70%, 80%, 90%, or 100% relative to a control siRNA molecule).

The disclosed dsRNA molecules may conveniently be delivered to a target cell or a target tissue through a number of delivery systems. For example, RNA may be delivered via electroporation, lipofection, calcium phosphate precipitation, plasmids, viral vectors that express the RNA, viral nucleic acids, phage nucleic acids, phages, cosmids, nanoparticles, or via transfer of genetic material in cells or carriers such as cationic liposomes. In one embodiment, transfection of RNA may employ viral vectors, chemical transfectants, or physico-mechanical methods such as electroporation and direct diffusion of DNA.

### Dinucleotide Repeat RNAs

The disclosed technology relates to the use of toxic RNA that is active in RNA interference (RNAi). In particular, the disclosed technology relates to dsRNAs comprising dinucleotide repeats and methods of using said dsRNAs in the treatment of disease.

In one aspect of the current disclosure, compositions are provided. In some embodiments, the compositions comprise: a dsRNA having a first strand, otherwise referred to as an “A” strand, and a second strand, otherwise referred to as a “B” strand,

### 5′-A01 A02 A03 A04 A05 A06 A07 A08 A09 A10 A11 A12 A13 A14 A15 A16 A17 A18 A19-3′

   | | | | | | | | | | | | | | | | | | |

### 3′-B19 B18 B17 B16 B15 B14 B13 B12 B11 B10 B09 B08 B07 B06 B05 B04 B03 B02 B01-5′

the dsRNA defined as follows: wherein A01 through A19 and B01 through B19 are any ribonucleotide selected from A, U, G, and C, provided that: (i) A01-A19 are complementary to B01-B19; (iii) the B strand comprises a dinucleotide repeat sequence (X₁X₂)ₙ, wherein X₁ and X₂ independently are selected from any ribonucleotide A, C, G, and U, and n is an integer from 3-9; or the A strand comprises a dinucleotide repeat sequence (X₁X₂)ₙ, wherein X₁ and X₂ independently are selected from any ribonucleotide A, C, G, and U, and n is an integer from 3-9. As used herein, “dinucleotide repeat” refers to a repeating set of two nucleotides.

The dinucleotide repeat may be selected from, for example: (AC)ₙ, (CA), (AG)ₙ, (GA)ₙ, (CU)ₙ, (UC)ₙ, (GU)ₙ, and (UG)ₙ. The dinucleotide repeat may also be selected from (UC)ₙ and (CU)ₙ.

Nucleic acid overhangs may improve the function of the disclosed dsRNAs, especially with regard to strand selection. Therefore, the disclosed dsRNAs may further comprises nucleic acid overhangs on the 3′ end of either the A, B, or both A and B strands. The nucleic acid overhangs may comprise deoxyadenosine (dA) and may comprise more than one dA covalently attached to the dsRNA with the formula (dA) m, e.g., wherein m=1, 2, 3, 4, 5, 6, etc.

Modification of nucleotides may further aid in strand selection by RISC or in protection from degradation by nucleases. For example, nucleotides may comprise 2′-O-methyl modifications or phosphorothioate modifications. In another example, the A01 and A02 ribonucleotides or the B01 and B02 ribonucleotides comprise 2′-O-methylation modifications. Suitably, the anti-sense strand, i.e., the strand that is complementary to the target RNA, e.g., mRNA, comprises 2′-O-methylation modifications.

### Pharmaceutical Compositions

In another aspect of the current disclosure, pharmaceutical compositions are provided. In some embodiments, the pharmaceutical compositions comprise: the disclosed compositions comprising dsRNAs; and a pharmaceutically acceptable carrier or excipient.

As used herein, the term “pharmaceutical composition” may be defined as a composition that includes a pharmacologically effective amount of a toxic RNA and/or extracellular particles comprising the toxic RNA and a pharmaceutically acceptable carrier for delivering the toxic RNA to target cells or target tissue. As used herein, the term “pharmaceutically acceptable carrier” refers to a carrier for administration of a therapeutic agent which facilitates the delivery of the therapeutic agent (e.g., a toxic RNA and/or extracellular particles comprising the toxic RNA) to target cells or target tissue. As used herein, the term “therapeutically effective amount” refers to that amount of a therapeutic agent that provides a therapeutic benefit in the treatment, prevention, or management of a disease or disorder (e.g., a cell proliferation disease or disorder such as cancer).

Suitable formulations of pharmaceutical compositions are known in the art. For example, pharmaceutical compositions may be formulated for intravenous, intramuscular, subcutaneous, intratumoral, oral, or parenteral administration.

### Methods of Treatment

In another aspect of the current disclosure, methods of treating a cell proliferative disease or disorder in a subject in need thereof are provided. In some embodiments, the methods comprise administering an effective amount of the pharmaceutical compositions of the instant disclosure to the subject treat the cell proliferative disease or disorder. The cell proliferative disease or disorder may be cancer and the cancer may be selected from, by way of example, but not by way of limitation, lung cancer, prostate cancer, ovarian cancer, colon cancer, and hepatocellular carcinoma.

In another aspect of the current disclosure, methods of killing a cancer cell are provided. In some embodiments, the methods comprise contacting the compositions of the instant disclosure to the cancer cell. The cancer cell may be, or may be derived from, for example, lung cancer, prostate cancer, ovarian cancer, or hepatocellular carcinoma.

The disclosed polynucleotides comprising a dsRNA sequence may, for example, be present as part of a siRNA or may be present as part of a shRNA, for example, wherein the 3′ of the A strand is linked via polynucleotides to the 5′ end of the B strand and/or wherein the 3′ end of the B strand is linked via polynucleotides to the 5′ end of the A strand, and the linking polynucleotide for a loop. The shRNA comprising the dsRNA sequence may be processed via Dicer to prepare an RNAi active siRNA.

The disclosed polynucleotides may also be expressed via an expression vector which optionally is inducible. For example, the disclosed polynucleotides may include shRNA that is expressed via an expression vector.

The disclosed expression vectors may be present in a cell such as a eukaryotic cell. In another example, the disclosed expression vectors may express a dual activity toxic shRNA and may be present in a eukaryotic cell which has been engineered to be deficient in a gene that is required for processing toxic shRNA to siRNA for RNA interference (RNAi). As such, the eukaryotic cell can be utilized to express the dual activity toxic shRNA and the eukaryotic cell will be resistant to the toxicity of the shRNA.

The disclosed polynucleotides and expression vectors may be formulated as pharmaceutical compositions comprising the polynucleotides and/or expression vectors and optionally comprising a pharmaceutically acceptable carrier, excipient, or diluent.

Also disclosed herein are extracellular vesicles comprising the disclosed polynucleotides. The disclosed extracellular vesicles may be produced and isolated from a eukaryotic cell that expresses the disclosed polynucleotides. For example, a eukaryotic cell which has been engineered to be deficient in a gene that is required for processing toxic shRNA to siRNA for RNA interference (RNAi) can be utilized to produce dual activity toxic shRNA as contemplated herein as well as extracellular vesicles (e.g., exosomes) that comprise the dual activity toxic shRNA as contemplated herein. The extracellular vesicles may be formulated as a pharmaceutical composition comprising the extracellular vesicles and optionally a pharmaceutically acceptable carrier, excipient, or diluent.

The disclosed polynucleotides, expression vectors, extracellular vesicles, and pharmaceutical compositions comprising the same may be administered in methods for treating a disease or disorder in a subject in need thereof. In some embodiments of the disclosed methods, the disease or disorder is a cell proliferative disease or disorder such as cancer. In particular, the disclosed polynucleotides, expression vectors, extracellular vesicles, and pharmaceutical compositions comprising the same may be administered to inhibit the growth of a cancer cell or to kill a cancer cell.

### EXAMPLES

The following Examples are illustrative and are not intended to limit the scope of the claimed subject matter.

### 1. Introduction

We previously demonstrated that a number of short double stranded RNAs based on trinucleotide repeat (TNR) sequences are super toxic to cancer cells through RNA interference (RNAi) (2, 3). The most toxic TNRs were based on CAG and CUG repeats. We determined this in an arrayed screen of all 64 possible TNRs converted to siRNAs with the TNR to be tested on the guide strand and the fully complimentary passenger strand disabled by adding a 2′-O-methylation at position 1 and 2 of the designated passenger strand (2). We went on to show that a CAG TNR based siRNA (siCAG) when delivered to a preclinical mouse model of ovarian cancer using HDL mimetic nanoparticles resulted in reduced tumor growth with no toxicity to the treated mice (2). siCAG killed cells by targeting CUG TNR containing genes present in human and mouse genomes.

2. Certain Dinucleotide Repeat (DNR) Based siRNAs are Super Toxic to Cancer Cells.

The human genome contains multiple low complexity repeat regions. Some of the most abundant repeat sequences are located in the ˜400 genomic clusters coding for ribosomal RNA (rRNA) (4). Much of these rDNA intergenic spacers are comprised of long simple dinucleotide repeat (DNR) sequences such as based on cytosine/thymine (CT) or adenosine/guanine (AG) repetitive sequences. These have often been viewed as “junk” DNA (5). Interestingly, these long DNRs are the only evolutionarily conserved regions between human and mouse rDNA in the inter-genic spacers. Recently, it was shown that these regions are actively transcribed in response to cellular stress suggesting that they could be functionally important (6). Similar to TNRs we posited the existence of gene regions that could give rise to DNR containing RNAs as well as genes that contained complementary DNRs that could be targeted by RNAi and induce cell death of cancer cells.

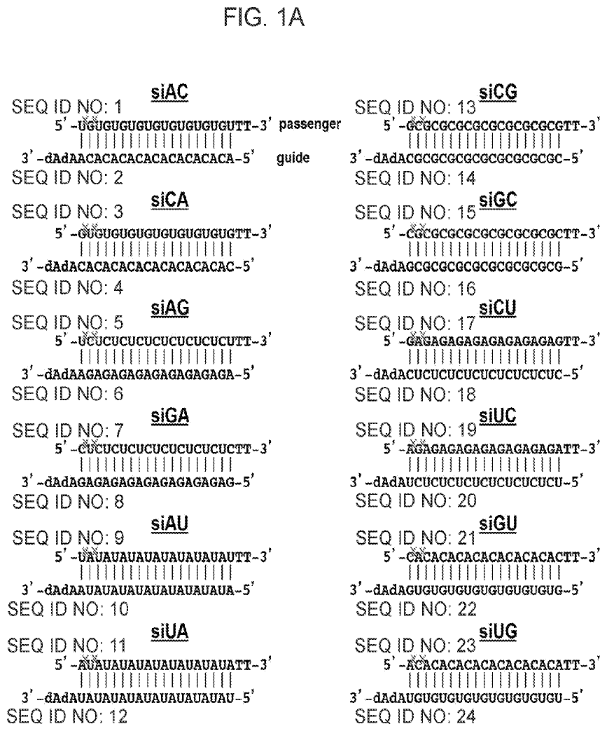

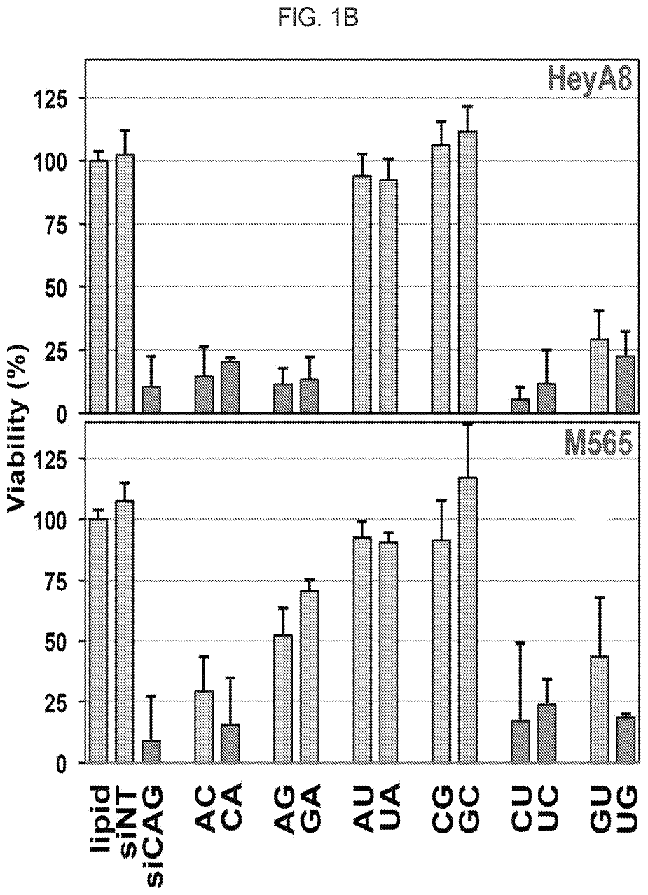

Similar to the screen of the 64 TNR based siRNAs we therefore performed an arrayed screen of the 12 different DNRs that contain more than one type of nucleotides (FIG. 1A). In fact the DNR screen, while never published or publicly disclosed, was part of the same screen in which we tested for the toxicity of all TNR based siRNAs (2). It was therefore performed exactly as previously described. In brief, siRNAs were transfected in a 384 well plate format in triplicate into a human (ovarian cancer cell line HeyA8) and a mouse (hepatocellular carcinoma cell line M656) cell line at 10 nM and cell survival (ATP assay) was quantified 96 hrs after transfection (FIG. 1B). Only one DNR based siRNA family (siCU/siUC) was super toxic (reduced cell viability >25%) to both cell lines suggesting evolutionary conservation. Assuming the toxicity was caused by RNAi this would suggest that CU based DNRs target complementary AG containing DNRs. Another highly toxic DNR was siCA/siAC.

It is possible that AU and GC based siRNAs were not toxic because AU rich siRNAs are not well loaded into the RNA induced silencing complex (RISC) because of weak base pairing and CG rich siRNAs are not well loaded because of base paring that is too strong (7) but that would need further analysis.

3. CU/UC Based siRNAs are Highly Toxic to Multiple Cancer Cells.

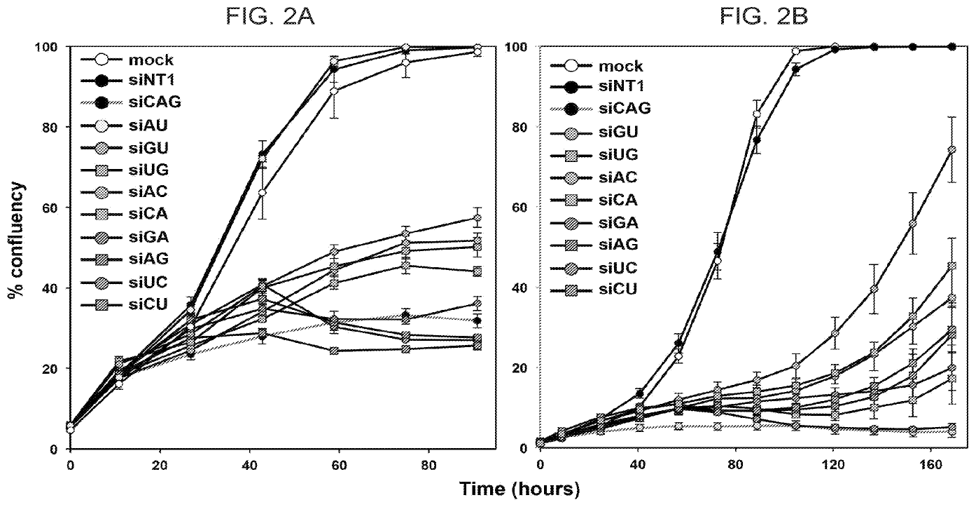

To determine which DNR based siRNAs are most toxic to cancer cells and how that compared to the potency of CAG TNR based siRNAs, we reverse transfected different DNR based siRNAs into HeyA8 cells at 10 nM and 1 nM (FIG. 2). Confirming the results of the screen CU and UC DNR based siRNAs were most toxic to the cells at about the same level as siCAG (FIG. 2A). This was followed by the toxicity of AG/GA based siRNAs. Interestingly most of the siRNAs including siCAG were even more toxic at 1 nM compared to 10 nM, a phenomenon we have observed before (unpublished observations). This could be due to an inhibitory effect of higher concentrations of these repeat-based siRNAs. At 1 nM CA/AC DNR based siRNAs were also highly toxic to the cells.

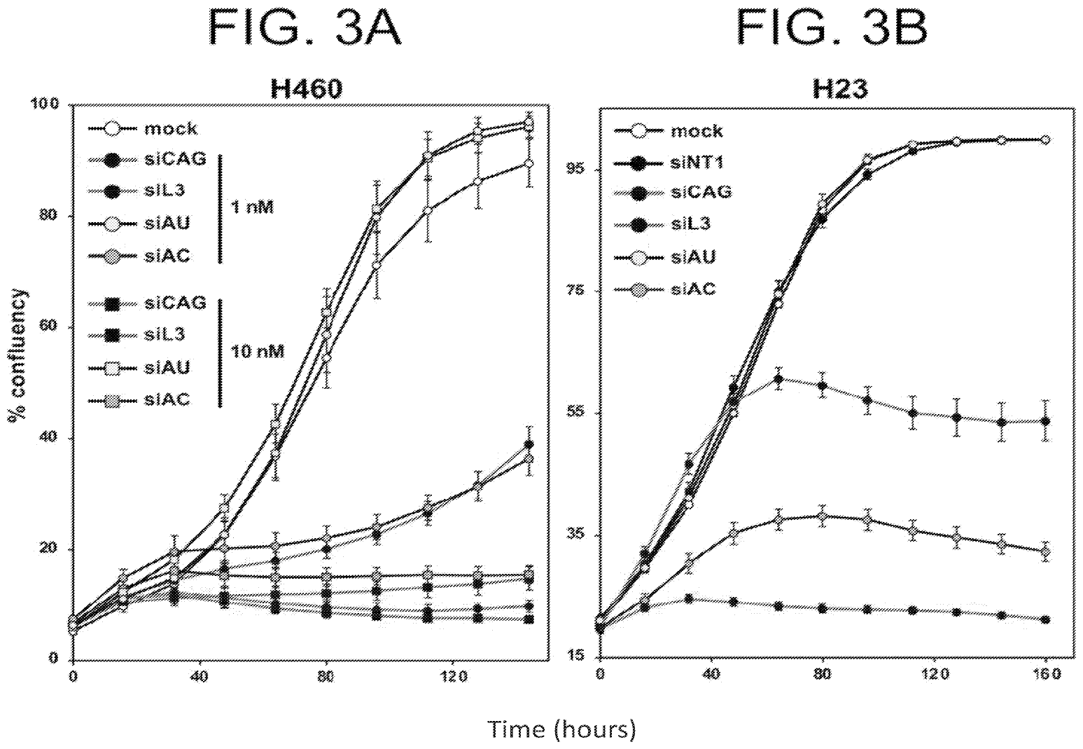

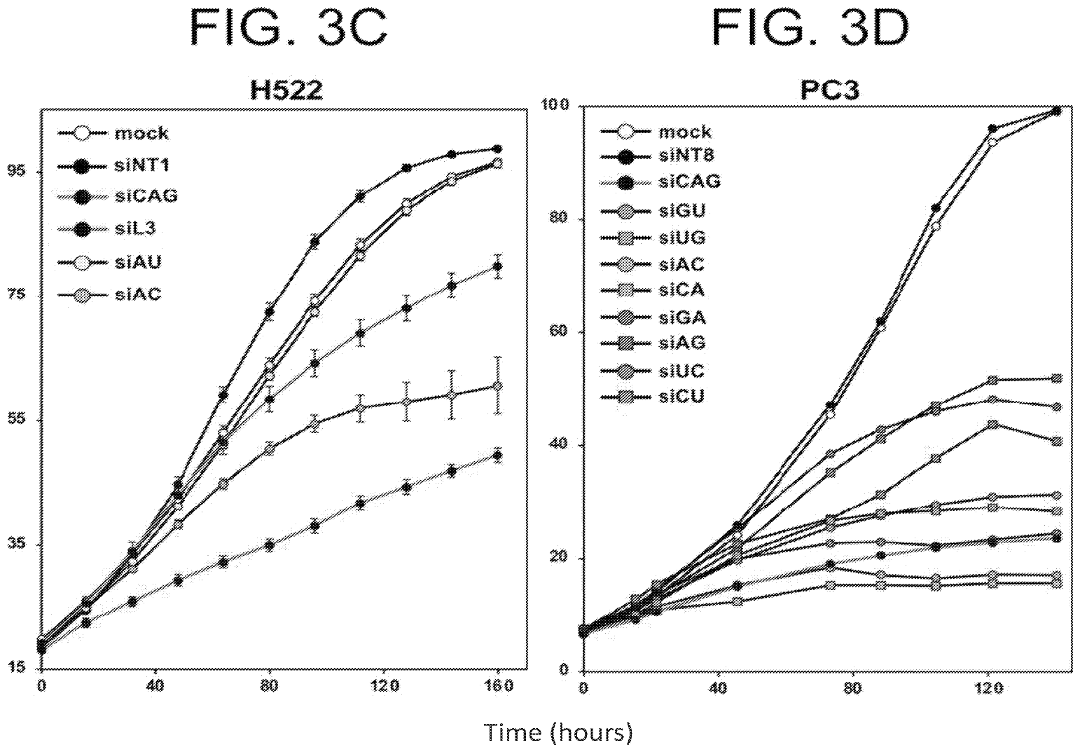

In a series of experiments, we tested multiple other cell lines representing different human cancers, including the lung cancer cell lines H460, H23, and H522 (FIGS. 3A-C), and the prostate cancer cell line PC3 (FIG. 3D). siAC was highly toxic to the lung cancer cells and it was the most toxic siRNA of all DNR in the prostate cancer cell line.

4. DNR Based siRNAs Kill Cells Through RNAi.

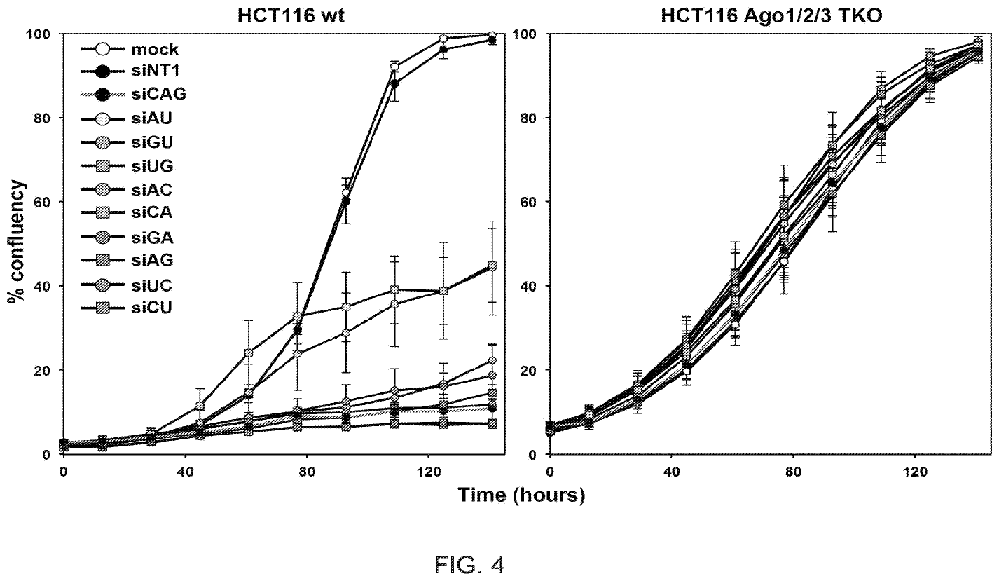

To determine whether any of the DNR based siRNAs killed cancer cells though RNAi, we tested all of them in the colon cancer cell line HCT116 lacking Argonaute (Ago) 1, 2, and 3, three major components of the execution machinery of RNAi, the RNA induced silencing complex (RISC). In addition to CU/UC DNR based siRNAs in this cell line siGA and siAG were the most toxic ones for wt HCT116 cells (FIG. 4). HCT116 are the only known cells in which all three main Ago genes can be deleted and cells remain viable (8). In parallel to wt cells we therefore tested Ago 1/2/3 triple knock out (TKO) cells. At 1 nM none of the siRNAs that were toxic to wt cells had any effect on the TKO cells, indicating that the toxicity of these siRNAs almost exclusively comes from RNAi and action of the RISC.

5. Frequency of DNRs in mRNAs in Human and Mouse.

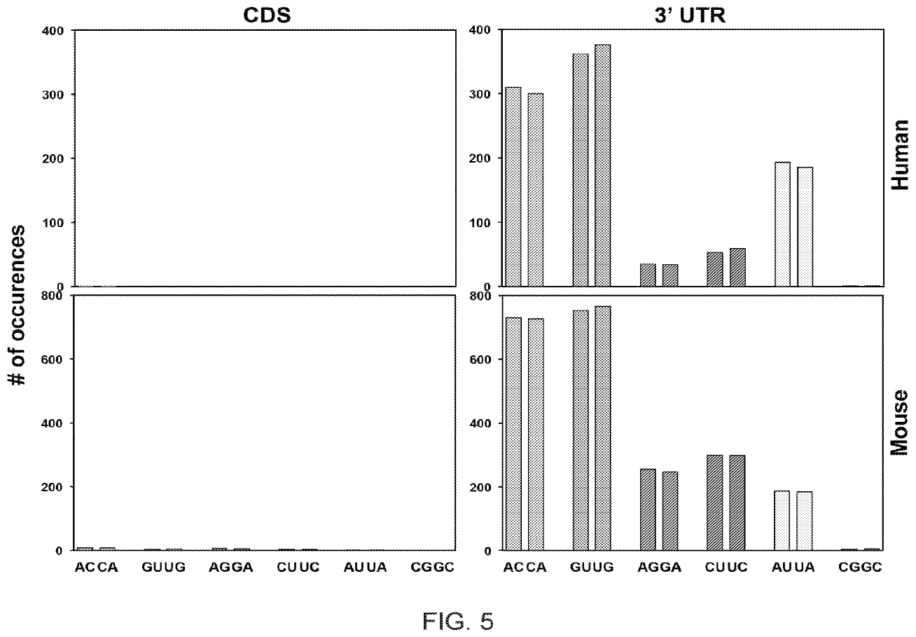

The involvement of RNAi in the cell death induced by DNR based siRNAs suggested that these siRNAs were toxic to cells by targeting mRNAs in the cells. We analyzed the human and mouse genome for the presence of DNRs of 19 nts or longer as these would most efficiently be targeted by the 19 nt long siRNAs (FIG. 5). An analysis of the open reading frame and the 3′ UTR of all human and mouse genes showed that virtually all DNRs are present in the 3′ UTRs with only a negligible number in the coding sequence (CDS). It is therefore likely that targeting of genes by DNR based siRNAs occurs in the 3′ UTR of genes.

6. Identification of Genes Silenced by siCU/siUC

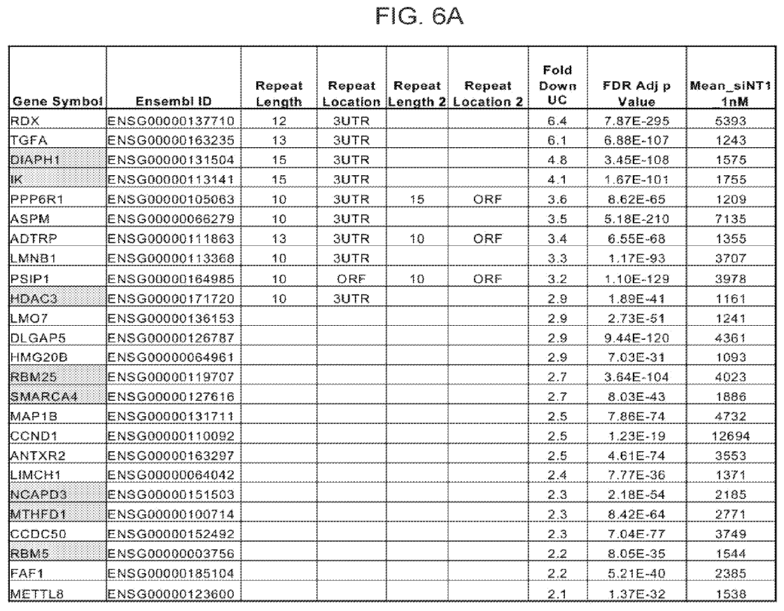

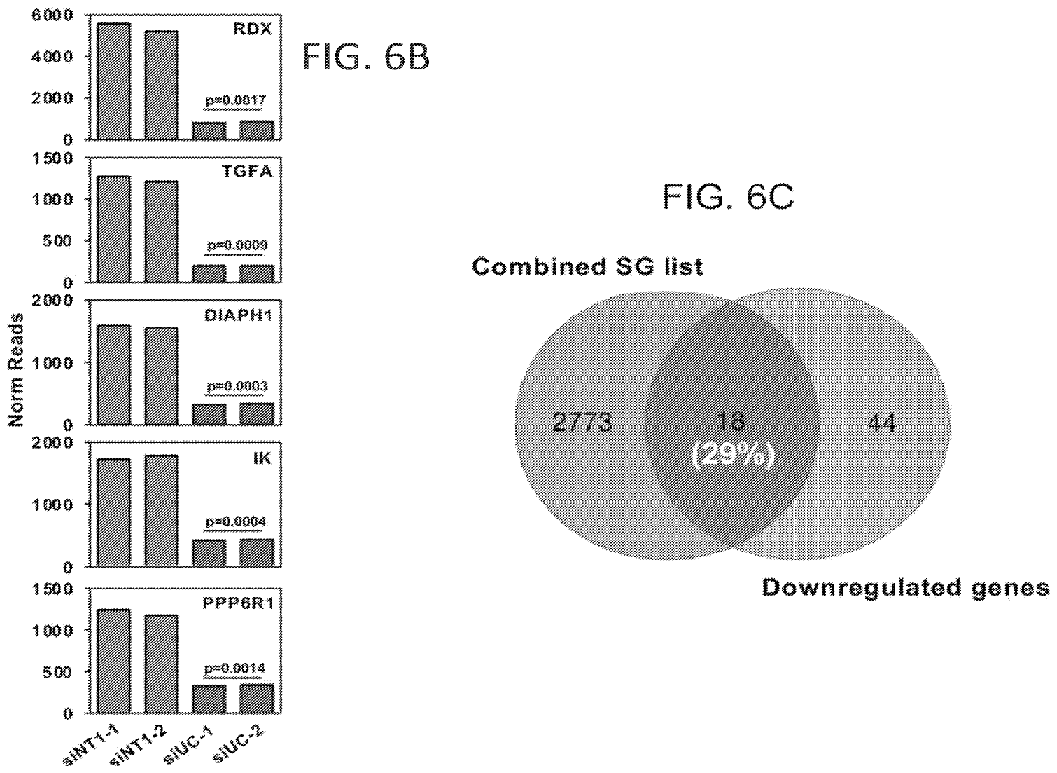

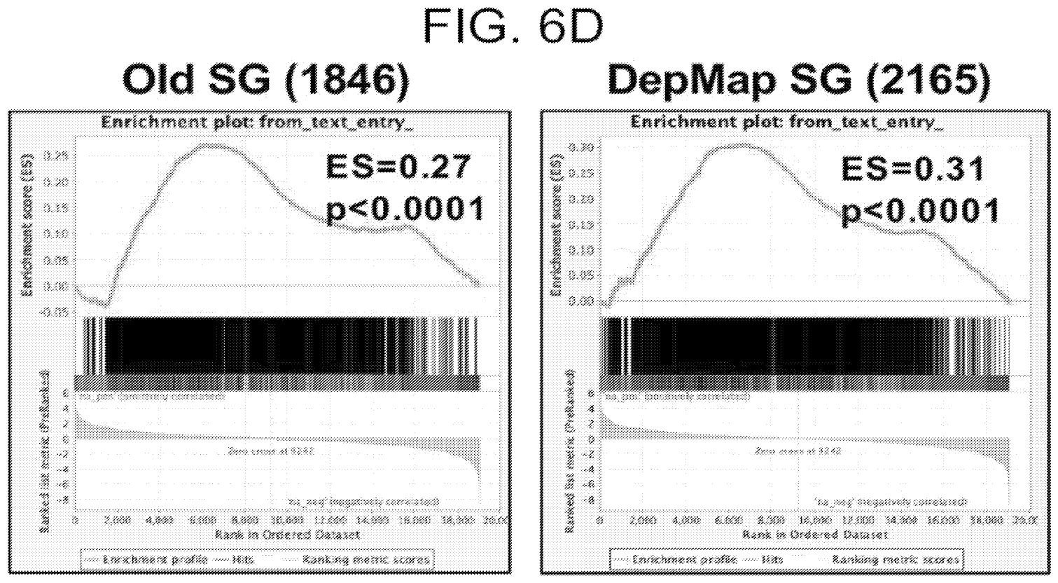

To identify possible targets of the DNR based siRNAs, we transfected HeyA8 cells with 1 nM of either the nontargeting siRNA siNT1, the nontoxic DNR based siRNA siAU or the two most toxic siRNAs siUC and siUC (FIG. 6). We then subjected the cells to an RNA Seq analysis and identified the genes that contained a reverse complementary DNR of 10 nts or longer anywhere in their mRNA, that were most downregulated by both siCU and siUC, and that had a minimum number of normalized reads (normalized to 1 million reads) of 1000 in the cells transfected with siNT1.

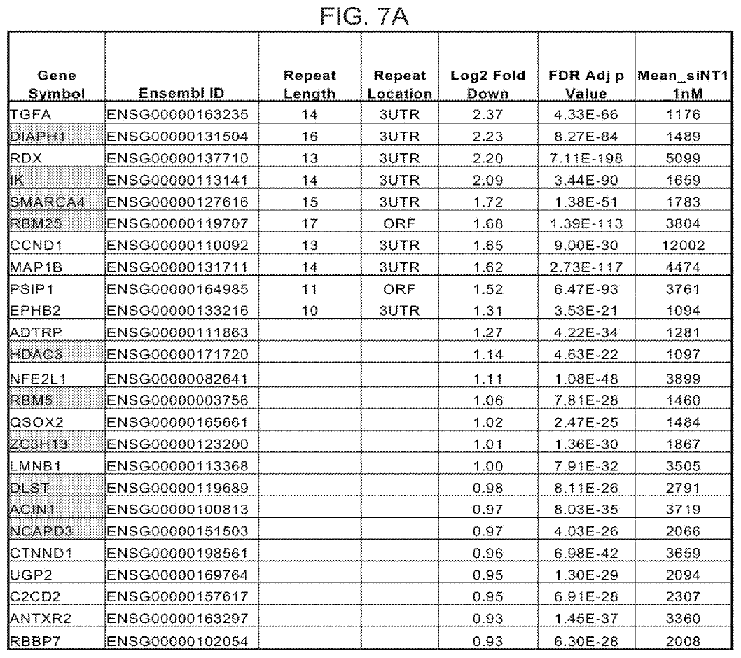

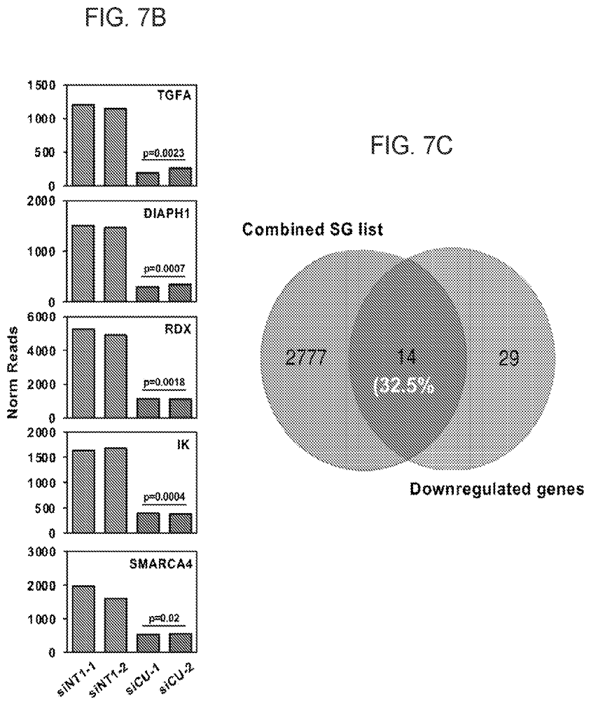

FIG. 6A shows the top 25 genes that were most downregulated in the cells, transfected with siUC compared to cells transfected with siNT1. Genes are highlighted that are part of either a curated list of survival genes (SGs) we previously published (1) or a list of 2165 essential genes that were identified by the Broad Institute by screening 1804 human cell lines (DepMAp.org), a total of 2791 SG genes. The length of the target AG DNR is given for the top 10 genes as well as its location in the mRNA. Some genes contain two targeted DNR motifs, one in the ORF and one in the 3′UTR. FIG. 6B shows the changes in read number in the duplicate data sets for the top 5 most highly downregulated genes. Consistently, 29% of the downregulated genes were in the list of SGs (FIG. 6C) and the enrichment of SGs in the downregulated genes was confirmed by a gene set enrichment analysis (GSEA) (FIG. 6D). These data suggest that cells died by a variant of death induced by survival gene elimination (DISE) (3, 9). The same analysis was repeated with cells transfected with either siNT1 or siCU (FIG. 7) with overall similar results. The GSEA did not reach statistical significance likely fewer genes (not shown) were downregulated compared to siUC transfected cells. However, of the 33 significantly expressed genes (>1000 read) that were at 1.5-fold downregulated >32% were SGs suggesting that again cells died by DISE (FIG. 7C).

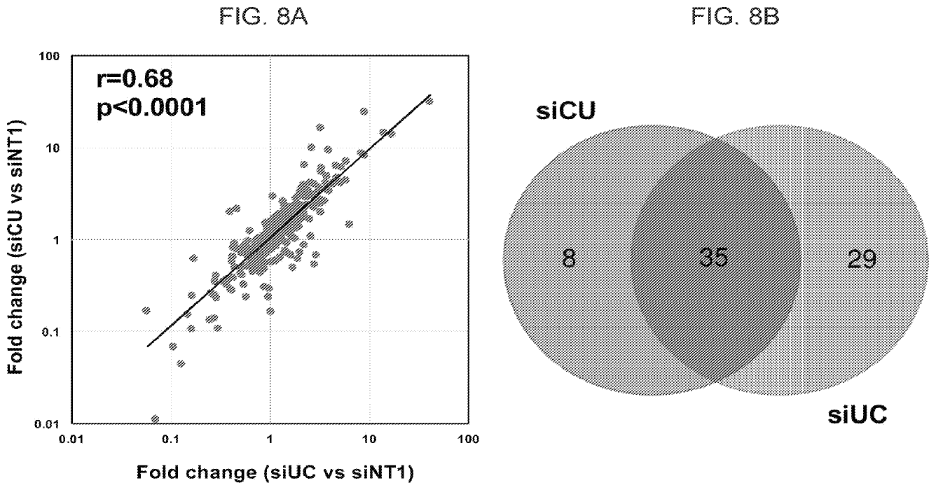

A comparison of the deregulated genes in cells transfected with siCU or siUC showed a wide overlap, consistent with their repetitive nature (FIG. 8). We interpret the somewhat larger number of complementary DNR containing genes targeted by siUC compared to siCU to the properties of siUC. With a U in the first position, it is expected to be loaded more efficiently into the RISC than siCU. Nevertheless, we found a high degree of overlap between the genes targeted by the two siDNRs.

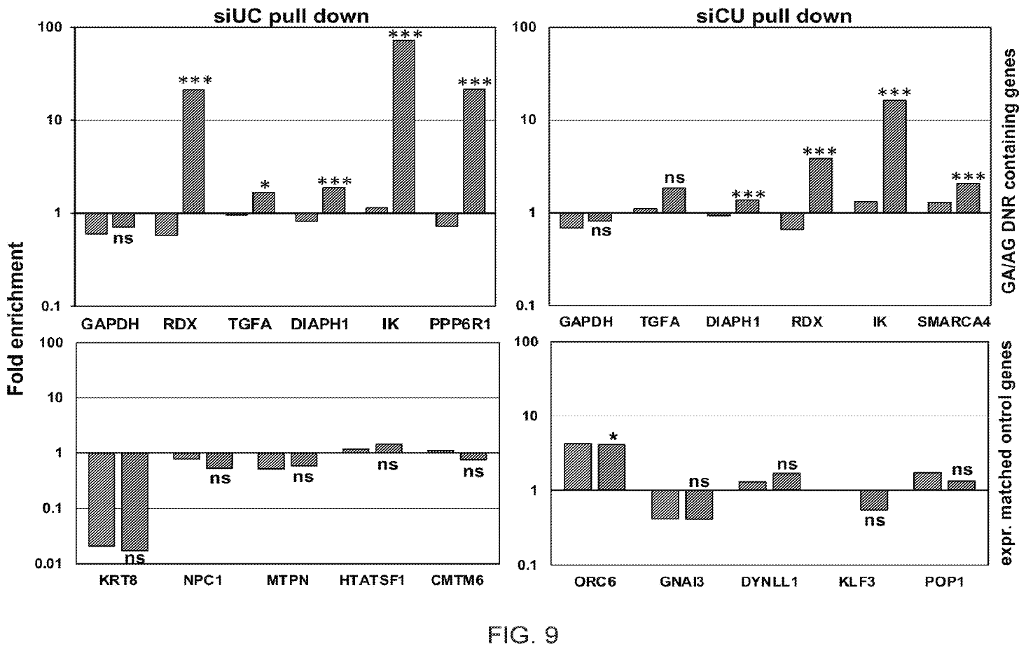

These data support our conclusion that siCU/siUC DNRs are toxic to cancer cells by silencing GA/AG DNR sequences that are critical for cell survival. To confirm that siUC/CU DNRs were indeed specifically targeting GA/GA DNR sequences, we pulled down biotinylated (Bi-)siUC or Bi-siCU transfected into HeyA8 cells (exactly as described previously (10) and subjected the precipitate to an RNAseq analysis. This analysis demonstrated that all of the top 5 most downregulated GA/GA DNR containing genes were indeed associated with the transfected siRNA (FIG. 9). No enrichment was found with the GAPDH control or with a set of control genes not containing targeted sites but expressed at similar levels. In summary, our data indicate that siRNAs comprised of either UC or CU DNRs kill cancer cells by targeting AG/GA DNR containing mostly located in the 3′UTR of the targeted genes and that very small amounts of these siRNAs are sufficient to kill cells.

In combination, based on our results the CU/UC and CA based siRNAs effectively kill cancer cells.

### REFERENCES

1. Putzbach W, Gao Q Q, Patel M, van Dongen S, Haluck-Kangas A, Sarshad A A, Bartom E, Kim K Y, Scholtens D M, Hafner M, Zhao J C, Murmann A E, Peter M E. (2017). Many si/shRNAs can kill cancer cells by targeting multiple survival genes through an off-target mechanism. eLife. 6: e29702.
2. Murmann A E, Gao Q Q, Putzbach W T, Patel M, Bartom E T, Law C Y, Bridgeman B, Chen S, McMahon K M, Thaxton C S, Peter M E. (2018). Small interfering RNAs based on huntingtin trinucleotide repeats are highly toxic to cancer cells. EMBO Rep. 19:e45336.
3. Murmann A E, Yu J, Opal P, Peter M E. (2018). Trinucleotide repeat expansion diseases, RNAi and cancer. Trends in Cancer. 4:684-700.
4. Warmerdam D O, Wolthuis R M F. (2019). Keeping ribosomal DNA intact: a repeating challenge. Chromosome Res. 27:57-72.
5. Smirnov E, Cmarko D, Mazel T, Hornacek M, Raska I. (2016). Nucleolar DNA: the host and the guests. Histochem Cell Biol. 145:359-72.
6. Wang M, Tao X, Jacob M D, Bennett C A, Ho J J D, Gonzalgo M L, Audas T E, Lee S. (2018). Stress-Induced Low Complexity RNA Activates Physiological Amyloidogenesis. Cell Rep. 24:1713-21 e4.
7. Chan C Y, Carmack C S, Long D D, Maliyekkel A, Shao Y, Roninson I B, Ding Y. (2009). A structural interpretation of the effect of GC-content on efficiency of RNA interference. BMC Bioinformatics. 10 Suppl 1: S33.
8. Chu Y, Kilikevicius A, Liu J, Johnson K C, Yokota S, Corey D R. (2020). Argonaute binding within 3′-untranslated regions poorly predicts gene repression. Nucleic Acids Res. 48:7439-53.
9. Putzbach W, Gao Q Q, Patel M, Haluck-Kangas A, Murmann A E, Peter M E. (2018). DISE-A Seed Dependent RNAi Off-Target Effect that Kills Cancer Cells. Trends in Cancer. 4:10-9.
10. Patel M, Bartom E T, Paudel B, Kocherginsky M, O'Shea K L, Murmann A E, Peter M E. (2022). Identification of the toxic 6mer seed consensus in human cancer cells. Sci Rep. 12:5130.

In the foregoing description, it will be readily apparent to one skilled in the art that varying substitutions and modifications may be made to the invention disclosed herein without departing from the scope and spirit of the invention. The invention illustratively described herein suitably may be practiced in the absence of any element or elements, limitation or limitations which is not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention. Thus, it should be understood that although the present invention has been illustrated by specific embodiments and optional features, modification and/or variation of the concepts herein disclosed may be resorted to by those skilled in the art, and that such modifications and variations are considered to be within the scope of this invention.

Citations to a number of patent and non-patent references are made herein. The cited references are incorporated by reference herein in their entireties. In the event that there is an inconsistency between a definition of a term in the specification as compared to a definition of the term in a cited reference, the term should be interpreted based on the definition in the specification.

1. A composition comprising:
a dsRNA having a first strand, otherwise referred to as an “A” strand, and a second strand, otherwise referred to as a “B” strand,
5′-A01 A02 A03 A04 A05 A06 A07 A08 A09 A10 A11 A12 A13 A14 A15 A16 A17 A18 A19-3′

| | | | | | | | | | | | | | | | | | |

3′-B19 B18 B17 B16 B15 B14 B13 B12 B11 B10 B09 B08 B07 B06 B05 B04 B03 B02 B01-5′
the dsRNA defined as follows: wherein
A01 through A19 and B01 through B19 are any ribonucleotide selected from A, U, G, and C, provided that:
(i) A01-A19 are complementary to B01-B19;
(ii) the B strand comprises a dinucleotide repeat sequence (X₁X₂)ₙ, wherein X₁ and X₂ independently are selected from any ribonucleotide A, C, G, and U, and n is an integer from 3-9; or the A strand comprises a dinucleotide repeat sequence (X₁X₂)ₙ, wherein X₁ and X₂ independently are selected from any ribonucleotide A, C, G, and U, and n is an integer from 3-9.

2. The composition of claim 1, wherein the dinucleotide repeat is selected from: (CU)ₙ, (UC)ₙ, (AC)ₙ, (CA)ₙ, (AG)ₙ, (GA)ₙ, (GU)ₙ, and (UG)ₙ.

3. The composition of claim 1, wherein the dinucleotide repeat is selected from (UC)ₙ and (CU)ₙ.

4. The composition of claim 1, wherein the dsRNA further comprises nucleic acid overhangs on the 3′ end of either the A, B, or both A and B strands.

5. The composition of claim 4, wherein the nucleic acid overhangs are (dA)ₘ.

6. The composition of claim 5, wherein m=2.

7. The composition of claim 6, wherein the overhang is only present on the B strand.

8. The composition of claim 1, wherein the composition comprises modified nucleotides.

9. The composition of claim 8, wherein the A01 and A02 ribonucleotides or the B01 and B02 ribonucleotides comprise 2′-O-methylation modifications.

10. The composition of claim 1, wherein the dsRNA comprises a sequence selected from the group consisting of: SEQ ID NO: 1-24.

11. The composition of claim 1, wherein the dsRNA comprises SEQ ID NO: 17 or 18.

12. The composition of claim 1, wherein the composition comprises SEQ ID NO: 19 or 20.

13. A pharmaceutical composition comprising the composition of claim 1 and a pharmaceutically acceptable carrier.

14. A method of treating a cell proliferative disease or disorder in a subject in need thereof comprising administering an effective amount of the pharmaceutical composition of claim 13 to the subject to treat the cell proliferative disease or disorder.

15. The method of claim 14, wherein the cell proliferative disease or disorder is cancer.

16. The method of claim 15, wherein the cancer is selected from lung cancer, prostate cancer, ovarian cancer, colon cancer, and hepatocellular carcinoma.

17. The method of claim 14, wherein administering comprises administering the pharmaceutical composition intravenously, intramuscularly, subcutaneously, intratumorally, or by inhalation.

18. The method of claim 14, wherein administering comprises administering the pharmaceutical composition intravenously.

19. A method of killing a cancer cell comprising contacting the composition of claim 1 to the cancer cell.

20. The method of claim 19, wherein the cancer cell is, or is derived from lung cancer, prostate cancer, ovarian cancer, colon cancer, or hepatocellular carcinoma.
